(19) United States
(12) Patent Application Publication
(10) Pub. No.: US 2006/0204549 A1
(43) Pub. Date: Sep. 14, 2006

(54) Method of improving nutrient utilisation by a mammal and a composition for use therein

(75) Inventors: Laura M'Rabet, Amersfoort (NL); Belinda Potappel-Van'Tland, Kootwijk (NL)

Correspondence Address: THE WEBB LAW FIRM, P.C., 700 KOPPERS BUILDING, 436 SEVENTH AVENUE, PITTSBURGH, PA, 15219, US

(73) Assignee: N.V. NUTRICIA, Zoetermeer (NL)

(21) Appl. No.: 10/541,566

(22) PCT Filed: Jan. 7, 2004
(86) PCT No.: PCT/NL04/00006

§ 371 Date: Apr. 7, 2006

(30) Foreign Application Priority Data
Jan. 7, 2003 (EP) 030750319

(51) Int. Cl. A61K 47/00; A61K 38/40

(52) U.S. Cl. 424/439; 514/6

(57) ABSTRACT

The invention relates to a method of stimulating nutrient utilization, especially through improved gastric emptying, comprising oral administration of a nutritional composition. The invention is concerned with a method of improving nutrient utilization by a mammal comprising orally administering a lactoferrin-containing nutritional composition to the mammal in an amount effective to reduce the residence time of nutrients in the stomach of the mammal, said composition exhibiting a weight ratio of lactoferrin to β-lactoglobulin that exceeds 1 to 15. The invention also relates to a pourable nutritional composition having a caloric content of at least 1.0 kcal/ml, a viscosity of 100 mPa.s or less at 21° C. and comprising: 50-79.6 wt. % water; at least 0.4 wt. % lactoferrin; 1-30 wt. % proteinaceous matter other than lactoferrin; 0.1-20 wt. % lipids; and 10-30 wt. % carbohydrates; wherein the combination of these components constitutes at least 80 wt. % of the composition and wherein the proteinaceous matter, lipids and/or carbohydrates together constitute at least 20 wt. % of the composition.

### TECHNICAL FIELD OF THE INVENTION

The present invention relates to a method of stimulating nutrient utilisation, especially through improved gastric emptying, comprising oral administration of a nutritional composition. Another aspect of the invention relates to a nutritional composition that is suitable for use in such a method.

### BACKGROUND OF THE INVENTION

Nutrient utilisation by mammals is affected by a variety of factors such as the mammal's ability to digest food and to absorb the nutrients that are liberated by the digestive process. In addition, nutrient utilisation is determined by a mammal's ability to ingest adequate amounts of food. The ability to ingest sufficient food may be affected adversely by a variety of conditions, such as nausea, vomiting and impaired gastrointestinal motility.

Conditions such as nausea and vomiting are often encountered in hospitalised patients, especially patients who undergo chemotherapy or radiotherapy, patients suffering from a gastrointestinal disorder and patients who have undergone surgery. It is crucial that such patients consume adequate amounts of nutrients, since malnutrition will adversely affect the recovery process. High caloric nutritional products that have been designed specifically to meet the nutritional needs of such patients are widely used. However, many of the aforementioned patients are unable to consume these products in adequate amounts, or if they do, they frequently throw up shortly after ingestion. The ability of these patients to consume food is closely related to the residence time of the food in the stomach. This is why nutritional products are preferentially supplied in liquid form. Unfortunately, however, even these liquid formulations do not offer sufficient solace for many patients who simply are unable to consume such products in a sufficient amount to meet their nutritional need.

Delayed gastric emptying is a gastrointestinal disorder that strongly affects nutrient utilisation and that is associated with symptoms such as nausea, vomiting, abdominal fullness or an early feeling of fullness when eating, distension or weight loss. Delayed gastric emptying may be caused by mechanical obstruction or by a gastric motility disorder. Diseases that have been associated with delayed gastric emptying include irritable bowel syndrome (IBS), functional dyspepsia, AIDS, diabetes mellitus, gastroesophageal reflux disease (GERD), anorexia nervosa, cachexia, surgery on the stomach or nervus vagus, smooth muscle disorders such as amyloidosis and scleroderma, nervous system diseases (including abdominal migraine and Parkinson's disease), metabolic diseases (including hypothyroidism), postviral syndromes and heartburn. Individuals suffering from delayed gastric emptying frequently develop symptoms associated with insufficient nutrient utilisation such as weight loss, infections and a general lack of energy.

As will be evident from the above, there is a need for a method of improving nutrient utilisation, particularly by decreasing the residence time of food in the stomach or, in other words, an accelerated gastric emptying into the gut (duodenum). There is also a need for an artificial nutritional composition which can be administered to individuals to improve their ability to utilise nutrients.

### SUMMARY OF THE INVENTION

The inventors have unexpectedly found that orally administered lactoferrin can have a stimulating effect on gastric motility without significantly decreasing the digestive function of the stomach. Based on this finding, the inventors have developed a method of improving nutrient utilisation by a mammal, said method comprising oral administration of lactoferrin in an amount effective to reduce the residence time of nutrients in the stomach.

Lactoferrin is an iron-binding glycoprotein closely related in structure to the serum iron transporting protein, transferrin.

Known medical applications of lactoferrin are largely based on the glycoprotein's host-protective and anti-inflammatory properties. The discovery that adequate oral dosages of lactoferrin can be used to stimulate gastric emptying could not be expected on the basis of the current understanding of the physiological effects of lactoferrin.

Lactoferrin naturally occurs in the milk of various mammals, including bovines. Thus, various dairy products contain detectable amounts of lactoferrin. Particularly high concentrations of lactoferrin are found in whey. Typically, bovine whey contains lactoferrin in an amount of no more than 4% by weight of β-lactoglobulin. The inventors have discovered that whey may be used as a suitable source of lactoferrin in accordance with the present invention, provided the weight ratio of lactoferrin to β-lactoglobulin in the final composition is substantially higher than those naturally found in whey, i.e. a weight ratio of lactoferrin to β-lactoglobulin that exceeds 1 to 15

FR-A 2 296 428 describes nutritional compositions containing lactoserum proteins. It is observed in the French application that lactoserum proteins can suitably be used in the treatment of maldigestion, digestive malabsorption and syndromes of malnutrition. The liquid nutritional compositions disclosed in the French application contain 10-15 g lactoserum protein per litre. Furthermore it is advocated therein that lactoferrin should be present in the nutritional composition in an amount of about 2 g per 100 g of total protein and B-lactoglobulin in an amount of about 47 g per 100 g of total protein.

WO 02/15719 is concerned with a nutritional supplement for convalescing patients, patients with limited appetite or those who have impaired ability to digest other sources of protein. The supplement comprises a protein source which provides at least about 8% total calories of the composition and which includes at least 50% by weight whey protein. It is stated in the application that it is believed that whey protein is rapidly emptied from the stomach and readily hydrolyzed and absorbed in the intestine. The PCT-application teaches liquid water based supplements wherein the protein source provides up to 20% of the total energy of the composition.

WO 98/50076 describes a method for treating or preventing insult-induced metabolic imbalance in animals by treating the gut, comprising administering to said animal a therapeutically or prophylactically effective amount of lactoferrin to control the generalized state of hypo- or hyper-activity. It is recommended in the application that the formulations employed in such a method contain 0.01-2 mg lactoferrin based on 1 ml or 1 g of carrier.

EP-A 0 295 009 teaches an infant formula which promotes gastrointestinal tract growth in newborn infans and newborn animals and which contains from about 0.1 to about 3 grams of lactoferrin per litre.

### DETAILED DESCRIPTION OF THE INVENTION

One aspect of the invention relates to a method of improving nutrient utilisation by a mammal comprising orally administering a lactoferrin containing nutritional composition to the mammal so as to provide lactoferrin to said mammal in an amount effective to reduce the residence time of nutrients in the stomach of the mammal, wherein said nutritional composition exhibiting a weight ratio of lactoferrin to β-lactoglobulin that exceeds 1 to 15. The present invention also encompasses the use of lactoferrin containing nutritional compositions that do not contain any β-lactoglobulin. Indeed, the weight ratio of lactoferrin to β-lactoglobulin in such lactoferrin containing compositions is infinitely high, i.e. well above the lower limit of 1 to 15.

The term “lactoferrin” as used in here, encompasses lactoferrin obtained from various, preferably natural sources, including e.g. bovine milk and plant sources such as soybeans. The term “lactoferrin” encompasses lactoferrin per se as well as nutritionally acceptable lactoferrin salts and precursors of lactoferrin that are converted into lactoferrin in vivo after oral administration.

Particularly good results can be obtained with the present method if the present nutritional composition contains at least 0.2 wt. %, preferably at least 0.4 wt. % lactoferrin. Most preferably, the present composition contains at least 0.6 wt. % lactoferrin.

In an especially preferred embodiment, the present nutritional composition is obtained by adding lactoferrin in a concentrated form, i.e. not, for example, in the form of whey or whey powder. The lactoferrin in the present composition may suitably be derived from whey, e.g. in the form of a whey fraction having an elevated level of lactoferrin, e.g. as demonstrated by an elevated ratio of lactoferrin to β-lactoglobulin. Preferably, the weight ratio of lactoferrin to β-lactoglobulin in the present composition exceeds 1 to 12, more preferably it exceeds 1 to 10, most preferably 1 to 8. In another preferred embodiment the weight ratio of lactoferrin to proteinaceous matter in the present composition exceed 25:1000, more preferably it exceeds 35:1000 and most preferably it exceeds 50:1000.

In another preferred embodiment, the present method comprises administering a composition containing at least 0.05 wt. % lactoferrin and 0-0.15 wt. % glycomacropeptide. Glycomacropeptide (GMP) is a hydrophilic peptide derived from casein. Ingestion of significant amounts of GMP may induce feelings of satiety, which effectively counteracts the advantageous effect resulting from the administration of lactoferrin. Consequently, the concentration of GMP in the present composition is advantageously kept below 0.15 wt. %.

GMP enhances the feeling of satiety upon entering the duodenum where it stimulates the synthesis and release of cholecystokinin (CCK). CCK slows down the overall digestive process which is perceived as a “fill”-feeling. To achieve improved nutrient utilisation, the composition that is administered in accordance with the present method should contain little to no GMP. In a particularly preferred embodiment, the lactoferrin containing composition contains less than 0.1 wt. % GMP, even more preferably less than 0.05 wt. % GMP.

Calculated on the proteinaceous matter in the present composition, the GMP content is preferably less than 5 wt. %, more preferably less than 1 wt. %, most preferably less than 0.2 wt. %.

Particularly good results can be obtained with the present method if lactoferrin is administered in a dosage of at least 0.1 g, preferably of at least 0.2 g. Usually, lactoferrin is administered in a dosage of up to 5 g. Preferably the lactoferrin dosage does not exceed 3 g. The term “dosage”, unless indicated otherwise, refers to the amount administered during a single administration event. In the case of the present method, such a single oral administration event may involve a number of bites, sips, swallows etc. Usually the administration event is completed within 40 minutes, preferably within 20 minutes.

In another preferred embodiment of the present method, lactoferrin is administered in a daily amount of at least 3.5 mg per kg of bodyweight, preferably in a daily amount of at least 7 mg per kg of bodyweight. Generally, the administered daily amount will not exceed 150 mg per kg of bodyweight, preferably said amount will not exceed 100 mg per kg of bodyweight.

The present method may be used to improve gastric emptying in all sorts of mammals. Preferably, the present method comprises administering lactoferrin to mammalian pets or farm animals such as cattle, sheep and pigs. The present method is particularly suited for improving nutrient utilisation in humans, especially non-infants.

In an especially preferred embodiment of the present method the lactoferrin is administered in the form of a nutritional composition that is pourable when it is orally administered. In another preferred embodiment said nutritional composition is pourable after a short residence time (e.g. not more than 5 minutes) in the stomach. A pourable nutritional composition offers the important advantage that is easily ingested and that it induces less satiety than solid or semi-solid compositions. Example of essentially non-pourable nutritional compositions that become pourable in the stomach include ice cream and gelled compositions whose gel network is decomposed under the acidic conditions that prevail in the stomach.

Typically, a pourable composition according to the invention exhibits a viscosity of not more than 100 mPa.s. Preferably, the pourable composition exhibits a viscosity of not more than 70 mPa.s, most preferably of not more than 40 mpa.s.

The nutritional composition employed in the present method preferably contains at least 0.1 wt. % lactoferrin and has a caloric content of at least 1 kcal/ml. The inclusion of an adequate amount of lactoferrin in a high caloric nutritional composition offers the advantage that the stimulating effect of the lactoferrin on gastric emptying will result in an accelerated passage of said high caloric nutritional composition through the stomach into the gastrointestinal tract. In addition, the risk of nausea and even vomiting resulting from the consumption of such a nutritional composition are reduced.

The present method is particularly advantageous if the nutritional composition has a relatively high caloric content. Consequently, in a preferred embodiment of the invention, the nutritional composition has a caloric content of at least 1.2 kcal/ml, preferably of at least 1.5 kcal/ml.

In a preferred embodiment of the invention, the present method comprises the co-administration of a carnitine source, particularly L-carnitine, a nutritionally acceptable salt of L-carnitine or a precursor of carnitine that is converted into L-carnitine in vivo after oral administration (e.g. L-carnitine, L-acetylcarnitine, L-isovalerylcarnitine, L-propionylcarnitine, alkanoyl carnitines, propionyl L-carnitine, valeryl L-carnitine, isovaleryl L-carnitine, acetyl L-carnitine, butyryl L-carnitine pharmacologically acceptable salts and mixtures thereof). Preferably the present method comprises co-administering a carnitine source in an amount equivalent to at least 10 mg L-carnitine, preferably in an amount equivelent to between 10 and 75 mg L-carnitine.L-carnitine is a water-soluble compound that is also referred to as vitamin BT. An important function of L-carnitine in the body is linked to the oxidation of fatty acids in mitochondria L-carnitine fulfils a critical role in the transportation of fatty acids into and out of mitochondria via the mitochondrial membrane. Thus, L-carnitine is an indispensable component for energy production in the body. L-carnitine is biologically synthesised from lysine and methionine in the body. However, the total metabolic turnover of L-carnitine exceeds the amount endogenously synthesised in the body. Therefore, the remaining metabolic turnover is derived from food. Carnitine deficiency is frequently observed in hospitalised patients.

In accordance with the present invention oral administration encompasses any form of administration that will effectively deposit the present composition into the stomach. Suitable examples of oral administration include oral consumption (ingestion) as well as tube feeding through mouth or nose. The present composition is usually administered in a single oral dosage of at least 20 ml, more preferably of at least 50 ml and most preferably of at least 100 ml. Generally the composition is administered in an amount that does not exceed 600 ml, more preferably does not exceed 400 ml and most preferably does not exceed 250 ml.

In order to achieve a significant improvement in nutrient utilisation it is advisable to administer the present composition at least once daily, preferably during a period of at least 3 consecutive days, more preferably during a period of at least 7 consecutive days.

The present method is particularly advantageous when applied to hospitalised patients, to mammals that suffer from catabolic stasis, from impaired gastric motility or from gastric obstruction. A special group of patients that may derive particular benefit from the present invention are patients who undergo chemotherapy.

The present method may also advantageously be used to improve nutrient utilisation in mammals whose ability to ingest, digest and absorb nutrients is not impaired. Accelerated gastric emptying in such mammals, e.g. athletes, is particularly advantageous shortly before or during physical exercise. Consequently, a special embodiment of the present invention the nutritional composition is consumed within 1 hour prior or during physical exercise.

Another aspect of the invention relates to a nutritional composition which is especially suitable for use in a method of improving nutrient utilisation as described herein before. More particularly, this aspect of the invention is concerned with a pourable nutritional composition that has a caloric content of at least 1.0 kcal/ml, a viscosity of 100 mPa.s or less at 21° C. and that comprises:
a 50-79.6 wt. % water;
b at least 0.4 wt. %, preferably 0.6-5 wt. % lactoferrin;
c 1-30 wt. %, preferably 1-20 wt. % proteinaceous matter other than lactoferrin;
d 0.1-20 wt. % lipids;
e 10-30 wt. % carbohydrates;
   wherein the combination of these components constitutes at least 80 wt. % of the composition and the proteinaceous matter, lipids and/or carbohydrates together constitute at least 20 wt. % of the composition. More preferably the constituents a. to e. together constitute at least 95 wt. %, most preferably at least 98 wt. % of the nutritional composition.

The term “proteinaceous matter” encompasses proteins, peptides as well as amino acids, but does not encompass lactoferrin.

The term “lipids” as used in here refers to triglycerides, diglycerides, monoglycerides, fatty acids and fosfolipids.

The term “carbohydrates” refers to mono-, di-, oligo- and polysaccharides. The nutritional composition may derive a pourable nature from the presence of essentially liquid components such as water and oil. Preferably the present product is water- and/or oil-continuous. Most preferably, the composition is water-continuous as the mouthfeel of water-continuous compositions is generally deemed to be superior to the mouthfeel of oil-continuous compositions.

The amount of proteinaceous matter present in the nutritional composition of the invention is preferably within the range of 7-25 wt. %. The incorporation of amounts of proteinaceous material in excess of 25 wt. % will normally result in a highly viscous product that will not pass easily through the stomach and that may induce feelings of satiety even after consumption of relatively minor quantities.

The proteinaceous matter contained in the present composition preferably comprises at least 80 wt. %, more preferably at least 90 wt. % of a combination of caseinate and whey protein. The weight ratio of caseinate to whey protein preferably is within the range of 1:10 and 10:1, more preferably within the range of 1:5 and 5:1. In a particularly preferred embodiment, the whey protein is obtained by ion exchange chromatography so as to remove a large fraction of the GMP that is naturally present in whey. The proteinaceous matter of the nutritional composition preferably contains less than 10% hydrolysed proteins.

In the present nutritional composition lipids and carbohydrates together preferably constitute at least 25 wt. %, more preferably at least 30 wt. % of the composition. In a particularly preferred embodiment the amount of lipids contained in the present composition is in the range of 0-30 wt. %, even more preferably in the range of 10-20 wt. %. The lipids contained in the present composition preferably consist for at least 80 wt. %, more preferably for at least 90 wt. % of triglycerides.

In another preferred embodiment the amount of carbohydrates is in the range of 10-40 wt. %, even more preferably in the range of 10-20 wt. %. These carbohydrates may comprise both digestible carbohydrates and indigestible carbohydrates such as dietary fibres. Preferably at least 60 wt. %, more preferably at least 80 wt. % of the carbohydrates are digestible carbohydrates.

The present nutritional composition advantageously comprises at least 5 wt. %, preferably 10-50 wt. % ω-3-fatty acids (including ω-3-fatty acid residues) by weight of the total amount of lipids. Omega-3 fatty acids are long chain aliphatic molecules beginning with a methyl group and ending with a carboxyl(ate) group. Omega-3 fatty acids contain a double bond in the third position from the methyl group. Two common, long chain omega-3 fatty acids are eicosapentanoic acid (20 carbons in length) and docosahexanoic acid (22 carbons in length). These are both found in fish oils

The inclusion of a significant fraction of the lipids in the form of ω-3-fatty acids in the present composition offers the advantage that these fatty acids produce less satiety than e.g. saturated fatty acids. Also, ω-3-fatty acids are believed to reinforce the stimulating effect of lactoferrin on gastric motility. Preferably the ω-3-fatty acid residues have a carbon chain length of 18-24 carbon atoms, even more preferably of 20-22 carbon atoms.

In addition to ω-3-fatty acid residues the present nutritional composition may also contain ω-6-fatty acid residues. In a preferred embodiment, the weight ratio of ω-6-fatty acid residues to ω-3-fatty acid residues is between 6:1 and 1:3.

In another preferred embodiment the composition comprises at least 5 wt. %, preferably 10-90 wt. % C₈-C₁₄ fatty acids (including C₈-C₁₄ fatty acid residues) by weight of the total amount of fatty acids. These medium chain fatty acids may advantageously be incorporated into the present composition to provide energy as fatty acids with a carbon chain length of 16 or less do not need carnitine for translocation across the mitochondrial membrane, which translocation is a a prequisite for the utilisation of these lipids in energy production. The people in need of the benefits of this invention are often carnitine deficient and therefore unable to fully utilise fatty acids with a carbon chain length of 16 or more.

In another preferred embodiment of the invention the composition contains at least 10 wt. %, preferably 20 wt % of carbohydrates with a glycemic index of less than 80, more preferably with a glycemic index of less than 75. Examples of suitable carbohydrates with such a low glycemic index include trehalose, maltodextrin, and palatinose. The incorporation of carbohydrates with a low glycemic index offers the advantage that they produce less satiety than carbohydrates with a high glycemic index.

The glycemic index is indicative of the effect of an ingested composition on blood glucose (blood sugar) levels. To determine the glycemic index of a composition, measured portions of the composition containing 50 grams of carbohydrate are fed to 10 healthy people after an overnight fast. Finger-prick blood samples are taken at 15-30 minute intervals over the next two hours. These blood samples are used to construct a blood sugar response curve for the two hour period. The area under the curve (AUC) is calculated and reflects the total rise in blood sugar (glucose) levels after eating the test food. The glycemic index rating (%) of the test composition is calculated by dividing the AUC for the test food by the AUC for the reference composition (containing 50 g of glucose) and multiplying by 100. The average of the glycemic index ratings from all ten subjects is published as the glycemic index of the test composition.

In order to prevent the occurrence of carnitine deficiency and the reduced energy production resulting from such deficiency, the present composition preferably contains at least 0.015 wt. %, more preferably between 0.020 and 0.045 wt. % carnitine.

The nutritional composition according to the invention is suitably compounded from a combination of nutritionally acceptable ingredients, for instance, by combining a source of lactoferrin (e.g. dry lactoferrin isolate) with water or a source of water (e.g. milk), as well as with the other components, i.e. lipids, carbohydrates and or proteinaceous material.

The present composition is preferably pasteurised or sterilised to prevent the risk of microbial spoilage. More preferably, the composition is pasteurised as sterilisation may lead to denaturation of the lactoferrin. Such denaturation is believed to adversely affect the stimulating effect of lactoferrin on gastric motility.

The present nutritional composition is advantageously packaged in the form units that represent a single dosage. Accordingly, another aspect of the invention relates to a packaged nutritional product that contains 20-400 ml, preferably of 100-250 ml of the nutritional composition as defined herein before. Such packaged nutritional products represent oral dosage units that may take the form of small bottles, containers or bags.

The invention is further illustrated by means of the following examples:

### EXAMPLES

### Example 1

The effect of lactoferrin on gastric emptying and nutritional intake in mammals is investigated in vivo using a model in which a decreased nutritional intake is induced.

Twenty four male Wistar rats with a body weight of 300-350 g are selected for the study and randomly assigned to 4 groups of each 6 rats. Two groups of rats receive continued infusion for 3 days of TNF-α (100 μg/kg bodyweight per day) while the remaining two groups serve as control groups. It is known that such a treatment will normally result in a substantially reduced feed intake.

Starting one day before the commencement of TNF infusion until the end of the experiment (total of 4 days) the different groups of rats receive the following:
Group A: Control group receiving feed
Group B: TNF group receiving feed
Group C: Control group receiving feed reinforced with the equivalent of a daily amount of 50 mg lactoferrin (lactoferrin replacing an equal amount of protein)
Group D: TNF group receiving feed reinforced with the equivalent of a daily amount of 50 mg lactoferrin (lactoferrin replacing an equal amount of protein)

Feed intake of the rats during the study is monitored. In addition, the speed of gastric emptying is monitored by the presence of a nutritional marker (e.g. CCK or insuline) in blood as measured at several timepoints.

Results show that the feed intake of groups A and C is comparable whereas the feed intake of group B is much lower than that of groups A and C. Feed intake of group D is found to be significantly higher than that of group B, which shows that oral administration of lactoferrin improved nutrient utilisation in rats whose nutrient utilisation is impaired by the infusion of TNF-α.

### Example 2

The effect of LF on gastric emptying and nutritional intake in patients is investigated in vivo by measuring gastric motility and gastric emptying in a population with impaired nutrient utilisation, gastric motility and gastric emptying such as diabetics, elderly, patients receiving chemotherapy or cancer patients suffering from GLP producing endocrine tumours.

Gastric motility, the frequency of gastric contractions, is measured by using cutaneous electrodes (electrogastrography, EGG). Gastric emptying is measured by using an [¹³C] octanoic acid breath test in which a nutritional bolus is supplemented containing labeled octanoic acid. The octanoic acid is immediately oxidised after gastric passage and ¹³CO₂ appears in the breath of the patient. The time lapsed between intake of the labelled bolus and appearance of ¹³CO₂ in the breath indicates the speed of gastric emptying.

Gastric emptying is followed by using a gamma-camera technique. Patients obtain a nutritional bolus of 150 ml, supplemented with a solid marker such as 20 MBq technetium-99m-labelled stannous colloid. The amount of solid marker detected by the camera indicates the speed of gastric emptying. After supplementing the patients with the nutritional bolus, nutritional utilisation is measured by questionnaires filled out by the patients.

Two groups of fifteen patients are enlisted for a study in which one group (A) receives a bolus of nutritional composition A and the other group (B) receives a bolus of a nutritional composition B which is identical to composition A, except that part of the proteinaceous matter has been replaced by a lactoferrin containing whey isolate.

The constituents of both compositions A and Bare listed in the following table:

| | Composition A | Composition B |
|---|---|---|
| Ingredient | (wt. %) | (wt. %) |
| Caseinate | 5.7 | 5.7 |
| Whey protein | 4.8 | 3.8 |
| Carbohydrate | 16.9 | 16.9 |
| Fat | | |
| 4% milk fat (ex whey) | 6.3 | 6.3 |
| 35% canola oil | | |
| 34% soybean oil | | |
| 15% MCT oil | | |
| 12% KD Pharma oil (>90% EPA) | | |
| L-carnitine (e.g. L-carnitine or | 0.04 | 0.04 |
| acetyl-L-carnitine from | | |
| Lonza, >90% purity) | | |
| Water | 67.26 | 67.26 |
| Lactoferrin (e.g. from DMV | 0 | 1 |
| international; ion exchange | | |
| chromatography bovine whey; | | |
| containing >90% Lactoferrin) | | |

Gastric motility, gastric emptying and nutritional utilisation are determined as described above.

Results show that patients belonging to group B have a better nutrient utilisation and are less prone to feelings of satiety than patients belonging to group A.

1-20. (canceled)

21. A method of preventing malnutrition as a result of insufficient food ingestion in a mammal in need thereof, said method comprising orally administering a lactoferrin-containing nutritional composition in an amount effective to reduce the residence time of nutrients in the stomach of the mammal, said lactoferrin-containing composition exhibiting a weight ratio of lactoferrin to β-lactoglobulin that exceeds 1 to 15 and containing 0.1-20 wt. % lipids, (ω-3-fatty acids (including ω-3-fatty acid residues) representing at least 5 wt. % of the total amount of lipids.

22. The method according to claim 21, wherein the lactoferrin-containing nutritional composition contains at least 0.2 wt. % lactoferrin.

23. The method according to claim 21, wherein the lactoferrin-containing nutritional composition contains at least 0.05 wt. % lactoferrin and 0-0.15 wt. % glycomacropeptide.

24. The method according to claim 21, wherein lactoferrin is administered in a dosage of at least 0.1 g.

25. The method according to claim 21, wherein a pourable nutritional composition having a caloric content of at least 1 kcal/ml and a viscosity of 100 mPa.s or less at 21° C. is administered.

26. The method according to claim 21, further comprising co-administering a carnitine source in an amount equivalent to at least 10 mg L-carnitine.

27. The method according to claim 21, wherein the lactoferrin-containing nutritional composition is administered in an amount of 20-400 ml.

28. The method according to claim 21, wherein the mammal is hospitalized patient or suffers from impaired gastric motility.

29. The method according to claim 21, wherein the mammal is a patient undergoing chemotherapy.

30. The method according to claim 21, wherein the lactoferrin-containing nutritional composition is consumed within 1 hour prior to or during physical exercise.

31. A pourable artificial nutritional composition having a caloric content of at least 1.0 kcal/ml and a viscosity of 100 mPa.s or less at 21° C., comprising:
a) 50-79.6 wt. % water;
b) at least 0.4 wt. % lactoferrin;
c) 1-30 wt. % proteinaceous matter other than lactoferrin;
d) 0.1-20 wt. % lipids; and
e) 10-30 wt. % carbohydrates;
wherein the combination of these components constitutes at least 80 wt. % of the composition, and the proteinaceous matter, lipids and/or carbohydrates together constitute at least 20 wt. % of the composition.

32. The nutritional composition according to claim 31, wherein the weight ratio of lactoferrin to β-lactoglobulin exceeds 1 to 15.

33. The nutritional composition according to claim 31, wherein not more than 10 wt. % of the proteinaceous matter is hydrolyzed protein.

34. The nutritional composition according to claim 31, wherein the lipids and the carbohydrates together constitute at least 13 wt. % of the composition.

35. The nutritional composition according to claim 31, wherein the nutritional composition contains at least 5 wt. % ω-3-fatty acids (including ω-3-fatty acid residues) by weight of the total amount of lipids.

36. The nutritional composition according to claim 31, wherein the nutritional composition contains ω-6-fatty acid residues and ω-3-fatty acid residues in a weight ratio between 6:1 and 1:3.

37. The nutritional composition according to claim 31, wherein the nutritional composition contains at least 5 wt. % C₈-C₁₄ fatty acids (including C₈-C₁₄ fatty acid residues) by weight of the total amount of fatty acids.

38. The nutritional composition according to claim 31, wherein the nutritional composition contains at least 10 wt. % of carbohydrates with a glycemic index of less than 80.

39. The nutritional composition according to claim 31, wherein the nutritional composition contains at least 0.015 wt. % carnitine.

40. A packaged artificial nutritional product comprising 20-400 ml of a nutritional composition according to claim 31.
